(19) United States
(12) Patent Application Publication
(10) Pub. No.: US 2023/0323273 A1
(43) Pub. Date: Oct. 12, 2023

(54) TEMPERATURE DETECTION DEVICE AND METHOD IN LAMP

(71) Applicant: Changzhou Trendi Medical Technology Co., Ltd, Changzhou City (CN)

(72) Inventors: Kwok Leung Patrick Kwan, Hong Kong (CN); Qiaoling Chen, Changzhou City (CN); Hui Qiao, Changzhou City (CN)

(21) Appl. No.: 17/963,826

(22) Filed: Oct. 11, 2022

(30) Foreign Application Priority Data
Feb. 23, 2022 (CN) 202210164408.7

(51) Int. Cl. G01N 27/18

(52) U.S. Cl. CPC C12M 41/18

(57) ABSTRACT

The present disclosure belongs to the technical field of temperature detection devices for microfluidic chips, specifically relates to a temperature detection device and method in loop mediated isothermal amplification (LAMP). The temperature detection device in the LAMP includes a temperature measurement mechanism, a heat conduction pad, a heating layer, and a thermal insulation layer which are disposed in sequence from top to bottom; the temperature measurement mechanism enters a corresponding temperature measurement chamber to collect corresponding working chamber temperature data, that is, the heating layer adjusts, according to the corresponding working chamber temperature data, heat generated by a corresponding heating source; the thermal insulation layer prevents the heating layer from downwards transmitting the heat such that the heats generated by the heating sources in the heating layer are all transmitted to corresponding heating regions through the heat conduction pad. In the present disclosure, the temperature measurement mechanism feeds back the working chamber temperature data serving as a temperature stabilization basis of the microfluidic chip, so that the temperature in a LAMP process can be accurately detected, and stable and efficient LAMP is guaranteed.

### TECHNICAL FIELD

The present disclosure belongs to the technical field of temperature detection devices for microfluidic chips, and particularly relates to a temperature detection device and method in LAMP.

### BACKGROUND

In a loop mediated isothermal amplification (LAMP) technology, it is necessary to provide a constant temperature environment for samples, primers, enzymes and other substances for amplification.

Meanwhile, in an amplification process, the temperature of the constant temperature environment needs to be detected. Traditionally, a thermometer is used for measurement. Since the distribution of heat in each area is not uniform, when a thermometer is used, on the one hand, detected temperature data is not accurate enough, and on the other hand, the thermometer needs to be inserted into a temperature measurement chamber for measurement, and values are read outside; and samples, primers, enzymes and other substances need to be amplified in a closed environment.

In this invention, a linear resistor can be used to directly perform temperature measurement in the temperature measurement chamber by directly measuring a temperature of a heat-sensitive electric or magnetic element. Therefore, there is an urgent need to develop a new temperature detection device and method in LAMP to solve the above problems.

### SUMMARY

The present disclosure aims to provide a temperature detection device and method in LAMP, so as to solve the problem of how to directly collect temperature data of a working chamber in a temperature measurement chamber as a temperature stabilization basis.

In order to solve the above technical problem, the present disclosure provides a temperature detection device in LAMP. The temperature detection device includes a temperature measurement mechanism, a heat conduction pad, a heating layer, and a thermal insulation layer which are disposed in sequence from top to bottom; the temperature measurement mechanism is electrically connected to the heating layer; at least two heating sources are arranged on the heating layer; heating regions corresponding to various heating sources are formed on the surface of the heat conduction pad; the working chambers in the microfluidic chip are aligned with the corresponding heating regions and placed on the heat conduction pad; each working chamber is provided with a corresponding temperature measurement chamber in parallel such that the temperature measurement mechanism enters the corresponding temperature measurement chamber to collect corresponding working chamber temperature data, that is, the heating layer adjusts heat generated by the corresponding heating source according to the corresponding working chamber temperature data; and the thermal insulation layer prevents the heating layer from downwards heat transmitting such that the heats generated by the heating sources in the heating layer are all transmitted to the corresponding heating regions through the heat conduction pad.

In one embodiment, the heating layer includes a microprocessor, a pulse width modulation (PWM) driving circuit that electrically connected to the microprocessor, a first heating plate, and a second heating plate; and the microprocessor drives, through the PWM driving circuit, the first heating plate and the second heating plate to perform heating such that the first heating plate and the second heating plate respectively form a 70° C. heating source and a 95° C. heating source, that is, a 70° C. heating region and a 95° C. heating region are formed at corresponding positions on the surface of the heat conduction pad, so as to heat the corresponding working chambers in the microfluidic chip on the heat conduction pad.

In one embodiment, the first heating plate and the second heating plate both adopt ceramic heating plates.

In one embodiment, the temperature measurement mechanism includes a first temperature sensor and a second temperature sensor which are electrically connected to the microprocessor; and a first probe of the first temperature sensor and a second probe of the second temperature sensor respectively extend into the corresponding temperature measurement chambers to collect the corresponding working chamber temperature data.

In one embodiment, the heat generated by the heating source=heat loss+ ambient temperature loss+ heat resistance.

In one embodiment, the ambient temperature loss is Q=cmΔT, where c is specific heat capacity; m is air mass; and ΔT is an air temperature rise.

In one embodiment, the heat conduction pad includes a low-heat-resistance heat conduction pad; and the low-heat-resistance heat conduction pad transmits the heat generated by each heating source.

In one embodiment, the thermal insulation layer includes an aerogel thermal insulation thin film; and the aerogel thermal insulation thin film prevents the heating layer from downwards transmitting the heat such that the heats generated by the various heating sources in the heating layer are all transmitted to the corresponding heating regions through the heat conduction pad.

In another aspect, the present disclosure provides a temperature detection method in LAMP. The temperature detection method includes: disposing a temperature measurement mechanism, a heat conduction pad, a heating layer, and a thermal insulation layer in sequence from top to bottom; arranging at least two heating sources on the heating layer, and forming heating regions corresponding to the various heating sources on a surface of the heat conduction pad; aligning various working chambers in a microfluidic chip with the corresponding heating regions and placing same on the heat conduction pad, wherein each working chamber is provided with a corresponding temperature measurement chamber in parallel such that the temperature measurement mechanism enters the corresponding temperature measurement chamber to collect corresponding working chamber temperature data; adjusting, through the heating layer according to the corresponding working chamber temperature data, heat generated by the corresponding heating source; and using the thermal insulation layer to prevent the heating layer from downwards transmitting the heat.

In one embodiment, the above-mentioned temperature detection device in LAMP is applicable to heating the microfluidic chip.

The present disclosure has the beneficial effects that the temperature measurement mechanism feeds back the working chamber temperature data serving as a temperature stabilization basis of the microfluidic chip, so that the temperature in a LAMP process can be accurately detected, and stable and efficient LAMP is guaranteed.

Other features and advantages of the present disclosure will be described in the following specification, and partly become obvious from the specification, or understood by implementing the present disclosure.

In order to make the above-mentioned objectives, features and advantages of the present disclosure more obvious and easier to understand, preferred embodiments are listed below, and are described in detail as follows in conjunction with the accompanying drawings.

### BRIEF DESCRIPTION OF THE DRAWINGS

To describe the specific implementation modes of the present disclosure or the technical solutions in the prior art more clearly, drawings required to be used in the specific implementation modes or the illustration of the prior art will be briefly introduced below. Apparently, the drawings in the illustration below are some implementation modes of the present disclosure. Those ordinarily skilled in the art can also obtain other drawings according to these drawings without doing creative work.

FIG. 1 is a structural diagram of a temperature detection device in LAMP of the present disclosure;

FIG. 2 is a structural diagram of a heat conduction pad of the present disclosure;

FIG. 3 is a structural diagram of a heating layer of the present disclosure;

FIG. 4 is a structural diagram of a microfluidic chip of the present disclosure;

FIG. 5 is a structural diagram of a temperature measurement cavity of the present disclosure; and

FIG. 6 is a schematic block diagram of a temperature detection device in LAMP of the present disclosure.

In the drawings:
temperature measurement mechanism 1, first temperature sensor 11, first probe 111, first thermal insulation hermetic connection part 112, second temperature sensor 12;
heat conduction pad 2, low-heat-resistance heat conduction pad 21, 70° C. heating region 22, 95° C. heating region 23;
heating layer 3, printed circuit board (PCB) 31, 70° C. heating source 32, 95° C. heating source 33;
thermal insulation layer 4, microfluidic chip 5, first working chamber 51, second working chamber 52, first temperature measurement chamber 53, and second temperature measurement chamber 54.

### DETAILED DESCRIPTION OF THE EMBODIMENTS

In order to make the objectives, technical solutions and advantages of the embodiments of the present disclosure clearer, the technical solution of the present disclosure will be described clearly and completely below in combination with the drawings. Obviously, the embodiments described are part of the embodiments of the present disclosure, not all the embodiments. All other embodiments obtained by those of ordinary skill in the art based on the embodiments in the present disclosure without creative work shall fall within the protection scope of the present disclosure.

### Embodiment 1

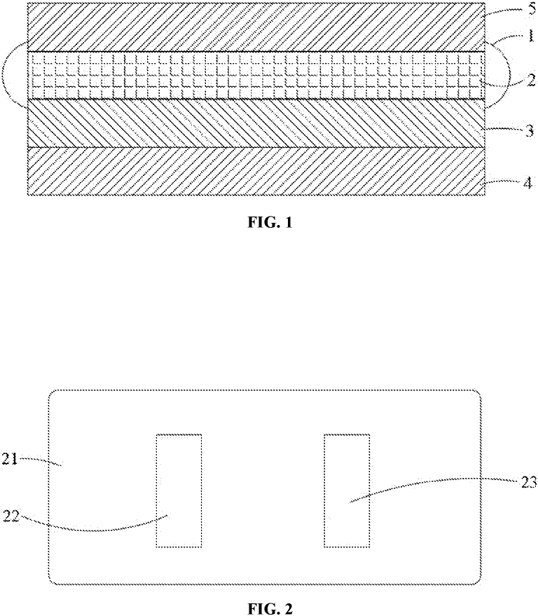

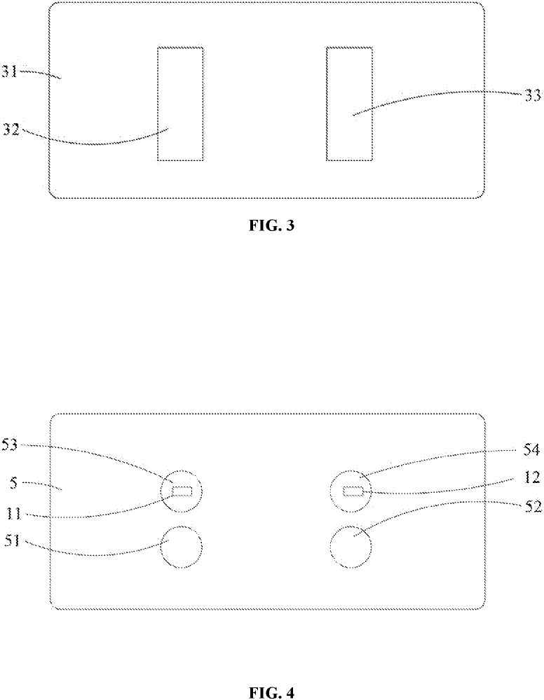

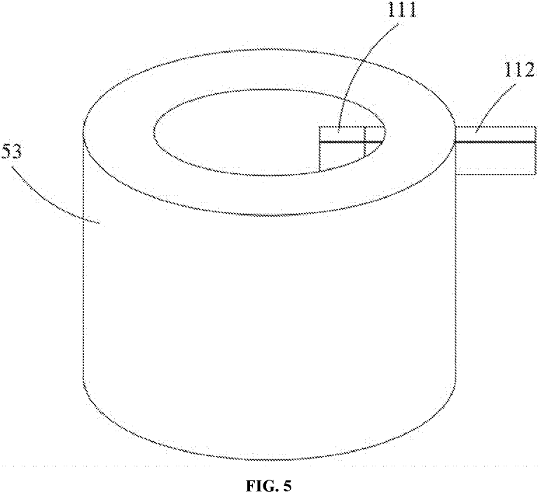

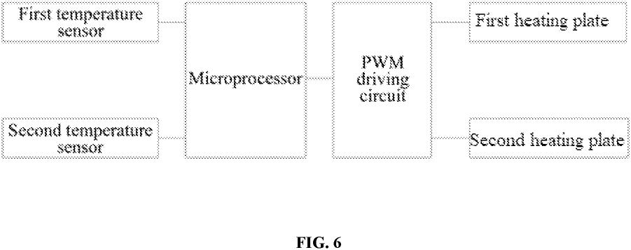

In this embodiment, as shown in FIG. 1 to FIG. 6, this embodiment provides a temperature detection device in LAMP. The temperature detection device includes a temperature measurement mechanism 1, a heat conduction pad 2, a heating layer 3, and a thermal insulation layer 4 which are disposed in sequence from top to bottom; the temperature measurement mechanism 1 is electrically connected to the heating layer 3; at least two heating sources are arranged on the heating layer 3; heating regions corresponding to the various heating sources are formed on a surface of the heat conduction pad 2; various working chambers in a microfluidic chip 5 are aligned with the corresponding heating regions and placed on the heat conduction pad 2; each working chamber is provided with a corresponding temperature measurement chamber in parallel such that the temperature measurement mechanism 1 enters the corresponding temperature measurement chamber to collect corresponding working chamber temperature data, that is, the heating layer 3 adjusts, according to the corresponding working chamber temperature data, heat generated by the corresponding heating source; and the thermal insulation layer 4 prevents the heating layer 3 from downwards transmitting the heat such that the heats generated by the various heating sources in the heating layer 3 are all transmitted to the corresponding heating regions through the heat conduction pad 2.

In this embodiment, the temperature measurement mechanism 1 feeds back the working chamber temperature data serving as a temperature stabilization basis of the microfluidic chip 5, so that the temperature in a LAMP process can be accurately detected, and stable and efficient LAMP is guaranteed. The working chamber temperature data is fed back through the temperature measurement mechanism 1; the heating layer 3 achieves constant-temperature control on the various heating regions through a PID feedback algorithm; a ceramic heating plate in the heating layer 3 can be applied to temperature control of the microfluidic chip 5; and due to an efficient heat conduction path composed of the low-heat-resistance heat conduction pad 2 and a pressure application method, the thermal insulation layer 4 adopts an aerogel thermal insulation thin film, so that the heat utilization efficiency is improved.

In this embodiment, the heating layer 3 includes a microprocessor, a PWM driving circuit electrically connected to the microprocessor, a first heating plate, and a second heating plate; and the microprocessor drives, through the PWM driving circuit, the first heating plate and the second heating plate to perform heating such that the first heating plate and the second heating plate respectively form a 70° C. heating source 32 and a 95° C. heating source 33, that is, a 70° C. heating region 22 and a 95° C. heating region 23 are formed at corresponding positions on the surface of the heat conduction pad 2, so as to heat the corresponding working chambers in the microfluidic chip 5 on the heat conduction pad 2.

In this embodiment, the 70° C. heating region 22 heats the first working chamber 51, and the 95° C. heating region 23 heats the second working chamber 52.

In this embodiment, the microprocessor, the PWM driving circuit, the first heating plate, and the second heating plate are all arranged on a PCB 31, and the PWM driving circuit is composed of a PWM driving chip serving as a core, and a peripheral circuit.

In this embodiment, as an optional implementation of the first heating plate and the second heating plate, the first heating plate and the second heating plate both adopt ceramic heating plates.

In this embodiment, the temperature measurement mechanism 1 includes a first temperature sensor 11 and a second temperature sensor 12 which are electrically connected to the microprocessor; and a first probe 111 of the first temperature sensor 11 and a second probe of the second temperature sensor 12 respectively extend into the corresponding temperature measurement chambers to collect the corresponding working chamber temperature data.

In this embodiment, the first temperature sensor 11 extends into the first temperature measurement chamber 53, and the second temperature sensor 12 extends into the second temperature measurement chamber 54.

In this embodiment, as an optional implementation of the first temperature sensor 11 and the second temperature sensor 12, the first temperature sensor 11 and the second temperature sensor 12 adopt PT1000 platinum resistors.

In this embodiment, a tail part of the first probe 111 is wrapped by a first thermal insulation hermetic connection part 112, so as to achieve thermal insulation and sealing and to ensure no impact to the collection work.

In this embodiment, a tail part of the second probe is wrapped by a second thermal insulation hermetic connection part, so as to achieve thermal insulation and sealing and to ensure no impact to the collection work.

In this embodiment, the heat generated by the heating source=heat loss+ ambient temperature loss+ heat resistance.

### Temperature Control Principle

The heat generated by the heating source= thermal space radiation + heat dissipation by a supporting frame (PMMA) of the thermal insulation layer 4 + a heat conduction medium (the corresponding heating plates + the heat conduction pad 2 + 0.1 mm PMMA) + heat dissipation by the microfluidic chip 5. The thermal space radiation is mainly affected by an ambient temperature and air flow. Since heating is carried out inside the equipment, the impact of the air flow is ignored, and the ambient temperature is variable. The heat dissipation by the supporting frame (PMMA) of the thermal insulation layer 4 is mainly affected by a material, an ambient temperature and air flow. Since the material is fixed, and the heating is carried out inside the equipment, the ambient temperature is a variable; and the thermal insulation layer 4 is placed between the corresponding heating plate and the PMMA support to minimize the heat loss. The heat transfer medium is affected by a PMMA material (constant) with a thickness of 0.1 mm in a heating range of the microfluidic chip 5, a material (constant) of the heat conduction pad 2, and a connection tightness (variable) between the microfluidic chip 5 and the corresponding heating plate; and the heat dissipation of the microfluidic chip 5 is affected by the ambient temperature (variable), air flow (constant), material (constant), and heated liquid (constant). Due to the above-mentioned variable analysis, the temperature control equivalent relationship can be simplified as:

The heat generated by the heating source = heat loss (constant) + ambient temperature loss (variable) + heat resistance (variable) between the microfluidic chip 5 and the corresponding heating plate.

The heat conduction pad 2 which is 1.0 mm thickness is used. By means of the heat conduction pad 2, according to a relationship between heat resistance and pressure, it can be seen that when a pressure is greater than 120 kPa, the heat resistance is basically kept being changed little, so that the heat resistance can be approximately a constant. Due to the above-mentioned variable analysis, the temperature control equivalent relationship can be further simplified as the heat generated by the heating source = heat loss (constant) + ambient temperature loss (variable) + heat resistance (constant). Temperature measurement is achieved by a symmetric structure method. One temperature measurement chamber is placed at a position parallel to the working chamber. In this way, the temperature of the temperature measurement chamber is closer to the temperature of the working chamber, and the temperature can be accurately measured by means of calibration and correction.

In this embodiment, the ambient temperature loss is Q=cmΔT, where c is specific heat capacity; m is air mass; and ΔT is an air temperature rise.

In this embodiment, the heat conduction pad 2 includes a low-heat-resistance heat conduction pad 21; and the low-heat-resistance heat conduction pad 21 transmits the heat generated by each heating source, so that an efficient heat conduction path can be formed.

In this embodiment, the thermal insulation layer 4 includes an aerogel thermal insulation thin film; and the aerogel thermal insulation thin film prevents the heating layer 3 from downwards transmitting the heat such that the heats generated by the various heating sources in the heating layer 3 are all transmitted to the corresponding heating regions through the heat conduction pad 2, so that the heat utilization efficiency can be improved.

### Embodiment 2

On the basis of Embodiment 1, this embodiment provides a temperature detection method in LAMP. The temperature detection method includes: disposing a temperature measurement mechanism 1, a heat conduction pad 2, a heating layer 3, and a thermal insulation layer 4 in sequence from top to bottom; arranging at least two heating sources on the heating layer 3, and forming heating regions corresponding to various heating sources on a surface of the heat conduction pad 2; aligning various working chambers in the microfluidic chip 5 with the corresponding heating regions, and placing same on the heat conduction pad 2, wherein each working chamber is provided with a corresponding temperature measurement chamber in parallel such that the temperature measurement mechanism 1 enters the corresponding temperature measurement chamber to collect corresponding working chamber temperature data; adjusting, through the heating layer 3 according to the corresponding working chamber temperature data, heat generated by the corresponding heating source; and using the thermal insulation layer 4 to prevent the heating layer 3 from downwards transmitting the heat.

In this embodiment, the temperature detection device in LAMP provided in Embodiment 1 is applicable to heating the microfluidic chip 5.

In this embodiment, the temperature detection device in LAMP includes a temperature measurement mechanism 1, a heat conduction pad 2, a heating layer 3, and a thermal insulation layer 4 which are disposed in sequence from top to bottom; the temperature measurement mechanism 1 is electrically connected to the heating layer 3; at least two heating sources are arranged on the heating layer 3; heating regions corresponding to various heating sources are formed on a surface of the heat conduction pad 2; various working chambers in a microfluidic chip 5 are aligned with the corresponding heating regions and placed on the heat conduction pad 2; each working chamber is provided with a corresponding temperature measurement chamber in parallel such that the temperature measurement mechanism 1 enters the corresponding temperature measurement chamber to collect corresponding working chamber temperature data, that is, the heating layer 3 adjusts, according to the corresponding working chamber temperature data, heat generated by the corresponding heating source; and the thermal insulation layer 4 prevents the heating layer 3 from downwards transmitting the heat such that the heats generated by the various heating sources in the heating layer 3 are all transmitted to the corresponding heating regions through the heat conduction pad 2.

In this embodiment, the working chamber temperature data is fed back through the temperature measurement mechanism 1; the heating layer 3 achieves constant-temperature control on the various heating regions through a PID feedback algorithm; a ceramic heating plate in the heating layer 3 can be applied to temperature control of the microfluidic chip 5; and due to an efficient heat conduction path composed of the low-heat-resistance heat conduction pad 2 and a pressure application method, the thermal insulation layer 4 adopts an aerogel thermal insulation thin film, so that the heat utilization efficiency is improved.

In this embodiment, the heating layer 3 includes a microprocessor, a PWM driving circuit electrically connected to the microprocessor, a first heating plate, and a second heating plate; and the microprocessor drives, through the PWM driving circuit, the first heating plate and the second heating plate to perform heating such that the first heating plate and the second heating plate respectively form a 70° C. heating source 32 and a 95° C. heating source 33, that is, a 70° C. heating region 22 and a 95° C. heating region 23 are formed at corresponding positions on the surface of the heat conduction pad 2, so as to heat the corresponding working chambers in the microfluidic chip 5 on the heat conduction pad 2.

In this embodiment, the 70° C. heating region 22 heats the first working chamber 51, and the 95° C. heating region 23 heats the second working chamber 52.

In this embodiment, the microprocessor, the PWM driving circuit, the first heating plate, and the second heating plate are all arranged on a PCB 31, and the PWM driving circuit is composed of a PWM driving chip serving as a core, and a peripheral circuit.

In this embodiment, as an optional implementation of the first heating plate and the second heating plate, the first heating plate and the second heating plate both adopt ceramic heating plates.

In this embodiment, the temperature measurement mechanism 1 includes a first temperature sensor 11 and a second temperature sensor 12 which are electrically connected to the microprocessor; and a first probe 111 of the first temperature sensor 11 and a second probe of the second temperature sensor 12 respectively extend into the corresponding temperature measurement chambers to collect the corresponding working chamber temperature data.

In this embodiment, the first temperature sensor 11 extends into the first temperature measurement chamber 53, and the second temperature sensor 12 extends into the second temperature measurement chamber 54.

In this embodiment, as an optional implementation of the first temperature sensor 11 and the second temperature sensor 12, the first temperature sensor 11 and the second temperature sensor 12 adopt PT1000 platinum resistors.

In this embodiment, a tail part of the first probe 111 is wrapped by a first thermal insulation hermetic connection part 112, so as to achieve thermal insulation and sealing and to ensure no impact to the collection work.

In this embodiment, a tail part of the second probe is wrapped by a second thermal insulation hermetic connection part, so as to achieve thermal insulation and sealing and to ensure no impact to the collection work.

In this embodiment, the heat generated by the heating source=heat loss+ ambient temperature loss+ heat resistance.

In this embodiment, the ambient temperature loss is Q=cmΔT, where c is specific heat capacity; m is air mass; and ΔT is an air temperature rise.

In this embodiment, the heat conduction pad 2 includes a low-heat-resistance heat conduction pad 21; and the low-heat-resistance heat conduction pad 21 transmits the heat generated by each heating source, so that an efficient heat conduction path can be formed.

In this embodiment, the thermal insulation layer 4 includes an aerogel thermal insulation thin film; and the aerogel thermal insulation thin film prevents the heating layer 3 from downwards transmitting the heat such that the heats generated by the various heating sources in the heating layer 3 are all transmitted to the corresponding heating regions through the heat conduction pad 2, so that the heat utilization efficiency can be improved.

TABLE I

Results of heating the second working chamber in the 95° C. heating region

| Purpose of the test | The target temperature 95+/-1° C. is stably maintained for 5 min | | |
|---|---|---|---|
| Test method | The PCB controls the ceramic heater to perform heating. | | |
| | The FLUKE 572 temperature measurer monitors the temperature of the chamber of the chip in real time; | | |
| | Data recording rule: every 1 min or if a temperature variation value exceeds 1° C.; Repeat for 3 times | | |
| Test data | The number of tests | Average temperature value °C (stabilized temperature) | Temperature fluctuation range °C (stabilized temperature) |
| | 1 | 95.2 | 0.19 |
| | 2 | 95.5 | 0.17 |
| | 3 | 95.4 | 0.25 |
| Test result | Repeat the test for 3 times: The temperature can be controlled within 95+/-1° C. and stably maintained for 5 min | | |

TABLE II

Results of heating the second working chamber in the 70° C. heating region

| Purpose of the test | The target temperature 70+/-1° C. is stably maintained for 30 min | | |
|---|---|---|---|
| Test method | The PCB controls the ceramic heater to perform heating. | | |
| | The FLUKE 572 temperature measurer monitors the temperature of the chamber of the chip in real time; | | |
| | Data recording rule: every 1 min or if a temperature variation value exceeds 1° C.; Repeat for 3 times | | |
| Test data | The number of tests | Average temperature value °C (stabilized temperature) | Temperature fluctuation range °C (stabilized temperature) |
| | 1 | 69.6 | 0.19 |
| | 2 | 69.7 | 0.18 |
| | 3 | 69.7 | 0.22 |
| Test result | Repeat the test for 3 times: The temperature can be controlled within 70+/-1° C. and stably maintained for 30 min | | |

To sum up, in the present disclosure, the working chamber temperature data is fed back through the temperature measurement mechanism; the heating layer achieves constant-temperature control on the various heating regions through a PID feedback algorithm; a ceramic heating plate in the heating layer can be applied to temperature control of the microfluidic chip; and due to an efficient heat conduction path composed of the low-heat-resistance heat conduction pad and a pressure application method, the thermal insulation layer adopts an aerogel thermal insulation thin film, so that the heat utilization efficiency is improved.

All the devices selected in the present application (components whose specific structures are not described) are general standard parts or parts known to those skilled in the art. Their structures and principles can be learned by those skilled in the art through technical manuals or conventional experimental methods.

In the description of the embodiments of the present disclosure, unless otherwise explicitly defined and defined, the terms “mounted”, “coupled” and “connected” shall be understood broadly, and may be, for example, fixedly connected, or detachably connected, or integrally connected, or mechanically connected, or electrically connected, or directly connected, or indirectly connected through an intermediate medium, or interconnection between two elements. Those of ordinary skill in the art can understand the specific meanings of the above terms in the present disclosure according to specific situations.

In the description of the present disclosure, it should be noted that orientations or positional relationships indicated by the terms “center”, “upper”, “lower”, “left”, “right”, “vertical”, “horizontal”, “inside”, “outside”, and the like are orientations or positional relationships as shown in the drawings, and are only for the purpose of facilitating and simplifying the description of the present disclosure instead of indicating or implying that devices or elements indicated must have particular orientations, and be constructed and operated in the particular orientations, so that these terms are not construed as limiting the present disclosure. In addition, the terms “first”, “second” and “third” are only for the purpose of description, and may not be understood as indicating or implying the relative importance.

In the several embodiments provided by the present disclosure, it should be understood that systems, devices, and methods disclosed may be implemented in other ways. The device embodiment described above is merely illustrative. For example, the division of units is a logical function division only. In actual implementation, there may be other division methods. For another example, multiple units or components may be combined or integrated into another system, or some features can be ignored, or not executed. From another point of view, the mutual coupling or direct coupling or communication connection shown or discussed may be indirect coupling or communication connection through some communication interfaces, apparatuses or units, and may be in electrical, mechanical or other forms.

The units described as separate components may or may not be physically separated, and the components displayed as units may or may not be physical units, that is, they may be located in one place, or they may be distributed on multiple network units. Part or all of the units may be selected according to actual needs to achieve the objectives of the solutions of the embodiments.

In addition, all functional units in all the embodiments of the present disclosure can be integrated into one processing unit, or each unit can physically exist alone, or two or more units can be integrated in one unit.

By taking the above-mentioned ideal embodiments according to the present disclosure as inspiration, through the above-mentioned description, relevant personnel can make various changes and modifications without departing from the scope of the technical idea of the present disclosure. The technical scope of the present disclosure is not limited to the content of the specification, and must be determined according to the scope of the claims.

What is claimed is:

1. A temperature detection device in loop mediated isothermal amplification (LAMP), comprising a temperature measurement mechanism, a heat conduction pad, a heating layer, and a thermal insulation layer which are disposed in sequence from top to bottom, wherein the temperature measurement mechanism is electrically connected to the heating layer; at least two heating sources are arranged on the heating layer; heating regions corresponding to various heating sources are formed on a surface of the heat conduction pad; various working chambers in a microfluidic chip are aligned with the corresponding heating regions and placed on the heat conduction pad; each working chamber is provided with a corresponding temperature measurement chamber in parallel such that the temperature measurement mechanism enters the corresponding temperature measurement chamber to collect corresponding working chamber temperature data, that is, the heating layer adjusts, according to the corresponding working chamber temperature data, heat generated by the corresponding heating source; the thermal insulation layer prevents the heating layer from downwards transmitting the heat such that the heats generated by the heating sources in the heating layer are all transmitted to the corresponding heating regions through the heat conduction pad; the heating layer comprises a microprocessor, a pulse width modulation (PWM) driving circuit electrically connected to the microprocessor, a first heating plate, and a second heating plate; the first heating plate and the second heating plate respectively form different temperature heating sources, so as to heat the corresponding working chambers in the microfluidic chip placed on the heat conduction pad.

2. The temperature detection device in the LAMP according to claim 1, wherein the microprocessor drives, through the PWM driving circuit, the first heating plate and the second heating plate to perform heating such that the first heating plate and the second heating plate respectively form a 70° C. heating source and a 95° C. heating source, that is, a 70° C. heating region and a 95° C. heating region are formed at corresponding positions on the surface of the heat conduction pad, so as to heat the corresponding working chambers in the microfluidic chip on the heat conduction pad.

3. The temperature detection device in the LAMP according to claim 2, wherein the first heating plate and the second heating plate both adopt ceramic heating plates.

4. The temperature detection device in the LAMP according to claim 2, wherein the temperature measurement mechanism comprises a first temperature sensor and a second temperature sensor which are electrically connected to the microprocessor; and a first probe of the first temperature sensor and a second probe of the second temperature sensor respectively extend into the corresponding temperature measurement chambers to collect the corresponding working chamber temperature data.

5. The temperature detection device in the LAMP according to claim 4, wherein the heat generated by the heating source=heat loss+ ambient temperature loss+ heat resistance.

6. The temperature detection device in the LAMP according to claim 5, wherein the ambient temperature loss is Q=cmΔT, where c is specific heat capacity; m is air mass; and ΔT is an air temperature rise.

7. The temperature detection device in the LAMP according to claim 1, wherein the heat conduction pad comprises a low-heat-resistance heat conduction pad; and the low-heat-resistance heat conduction pad transmits the heat generated by each heating source.

8. The temperature detection device in the LAMP according to claim 1, wherein the thermal insulation layer comprises an aerogel thermal insulation thin film; and the aerogel thermal insulation thin film prevents the heating layer from downwards transmitting the heat such that the heats generated by the various heating sources in the heating layer are all transmitted to the corresponding heating regions through the heat conduction pad.

9. A temperature detection method in LAMP, comprising: disposing a temperature measurement mechanism, a heat conduction pad, a heating layer, and a thermal insulation layer in sequence from top to bottom; arranging at least two heating sources on the heating layer, and forming heating regions corresponding to various heating sources on a surface of the heat conduction pad; aligning various working chambers in a microfluidic chip with the corresponding heating regions and placing same on the heat conduction pad, wherein each working chamber is provided with a corresponding temperature measurement chamber in parallel such that the temperature measurement mechanism enters the corresponding temperature measurement chamber to collect corresponding working chamber temperature data; adjusting, through the heating layer according to the corresponding working chamber temperature data, heat generated by the corresponding heating source; and using the thermal insulation layer to prevent the heating layer from downwards transmitting the heat, wherein the temperature detection method is applicable to heating the microfluidic chip using the temperature detection device for a microfluidic chip in LAMP according to claim 1.
